(19) United States
(12) Patent Application Publication
(10) Pub. No.: US 2010/0105628 A1
(43) Pub. Date: Apr. 29, 2010

(54) REDUCING POST-OPERATIVE ADHESION FORMATION WITH INTRAPERITONEAL GLUTAMINE

(75) Inventors: Adebola O.E. Obayan, Saskatoon (CA); George Kiremu Mutwiri, Saskatoon (CA)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITY OF SASKATCHEWAN, Saskatoon, Saskatchewan (CA)

(21) Appl. No.: 12/526,011

(22) PCT Filed: Feb. 14, 2008
(86) PCT No.: PCT/CA2008/000286

§ 371 Date: Dec. 8, 2009

(30) Foreign Application Priority Data
Feb. 15, 2007 (CA) 2578647

(51) Int. Cl. A61K 31/195; A61K 47/48; A61P 41/00

(52) U.S. Cl. 514/19; 514/563; 562/563

(57) ABSTRACT

Glutamine source formulations that have release profiles that provide for sustained release via intraperitoneal administration of glutamine reduces post-operative adhesion formation.

### BACKGROUND OF THE INVENTION

Adhesions are abnormal deposits of fibrous tissue that form within the peritoneal cavity. Abdominal adhesions are a common cause of small bowel obstruction and female infertility [1-3]. Adhesion formation occurs after any surgical procedure. However, it is extremely common after abdominal and pelvic operations and remains a source of considerable morbidity. The incidence ranges from about 67%-93% after general surgical abdominal operations and up to about 97% after open gynecologic pelvic procedures [4, 5]. In clinical and autopsy studies of patients who had prior laparotomy, the incidence of intra-abdominal adhesions was about 70-90% [6]. Use of both absorbable polyglycolic acid meshes and non-absorbable polypropylene meshes as reinforcing materials in surgery is associated with a high incidence of adhesion formation [6a, 6b].

Factors associated with the formation of post-surgical adhesions include trauma, thermal injury, infection, ischemia and foreign bodies. Other factors associated with adhesion formation include tight suturing where tension within the sutured peritoneum produces ischemia and abrasion. Exposure to foreign bodies such as talc and powders from the gloves, lint from abdominal packs or disposable papers items may also contribute to the formation of adhesions [7-9]. Neutropenia is associated with lower rates of adhesion and may play a role in modulating post-operative adhesion [10].

The peritoneum is composed of two mesothelial sheets that enclose predominantly adipocytes embedded in loose connective tissue, and also aggregates of mononuclear phagocytic cells. The greater omentum is the largest part of the peritoneum with the size varying from 300 gm to 2000 gm and a surface area of 300 cm² to 1500 cm². The omentum has a rich vascular supply with numerous characteristic capillary convolutions that are termed omental glomeruli due to their similarity with renal glomeruli. These capillary beds lie directly under the mesothelium [11]. Adhesions are formed as a result of fibrous repair of peritoneal injury mostly after surgery.

Milky spots develop as specific structures in the greater omentum of the peritoneum between the 20th and 35th week of gestation [12]. They are corpuscles found in the omental glumeruli measuring 0.1-2 mm in size, hardly visible to the naked eye, and under low magnification look like tufts of cotton wool [13, 14]. Milky spots are characterized by a permanent glomus pattern of vascular structure, specific cellular population and a specialized mesothelial lining. In humans, milky spots comprise of macrophages (70%), B-lymphocytes (10%), T-lymphocytes (10%), mast cells, and stromal cells. The mean number of cells in one milky spot is approximately 600 [15]. The number of milky spots is highest in infancy and gradually decreases with age [12]. The activation of milky spot, which occurs within 6 hours of abdominal surgery plays a role in adhesion formation [16].

The macrophages in the mature omentum are essentially scavengers. They appear to differentiate from monocytic precursors in the milky spots and are not dependent on precursors derived from the bone marrow [17]. They are dendritic in shape and have marked phagocytic abilities. They avidly phagocytose intraperitoneally injected carbon particles and bacteria. When activated, the macrophage precursors in the milky spots proliferate, migrate to the mesothelial surface, and transform into dendritic-shaped macrophages. Following surgery, macrophages increase in number and change function which are different from the resident macrophages and secrete variable substances including cycloxygenase and lipoxygenes metabolites, plasminogen activator, plasminogen activator inhibitor (PAI) etc [9, 18]. These macrophages recruit new mesothelial cells that proliferate forming islands in the injured areas resulting in peritoneal remesothelialization. Following stimulation of the milky spot there is an increased microvascular permeability to fluid, neutrophils, monocytes and fibrin deposits within the connective tissue matrix of milky spots, and subsequent increased cellular migration across the mesothelial lining into the peritoneal cavity [19].

Adhesion formation begins with injury inflicted on the peritoneum whether by an injurious stimuli including bacterial, chemical toxicity, ischemia, mechanical or simply drying from exposure [16, 25]. The injury leads to an inflammatory response, which progresses to fibrin deposition and subsequent fibrinous adhesion. If the fibrinous adhesion is not degraded within the first days of injury, reparative cells including fibroblasts are propagated into the fibrin matrix turning it into permanent fibrous adhesion. This process is completed within a week of the injury. The balance of fibrin deposition and breakdown is therefore crucial in the early phase of peritoneal repair and adhesion formation [25-27]. Peritoneal macrophages may be involved in regulation of plasmin activity in the peritoneal cavity [28], and thus a role in adhesion formation [29].

Various methods of adhesion prevention and treatment have been employed, including prevention of fibrin deposition in the peritoneal exudate, reduction of local tissue inflammation, and removal of fibrin deposits. Most of the existing methods inhibit one of these categories and yet have limited success. Implants in the form of resorbable fabrics or membranes (which are thought to act as macrophage barriers) as well as gels formed of biocompatible materials have also been employed to reduce formation of adhesions. Examples are the products sold under the trademarks INTERCEED and SEPRAFILM.

Glutamine is a conditional essential amino acid which the body is unable to synthesize in sufficient quantities under certain physiologic circumstances [30, 31] such as major surgery, shock, traumatic injury and severe sepsis. A decrease in extracellular glutamine impairs the function of macrophages and other immune cells, resulting in increased protein degradation from skeletal muscle [20]. Macrophages are extremely active cells (10 times per minute based on ATP turnover and 5 times a minute based on oxygen consumption) with a high capacity to take up glutamine and ‘trap it’ as glutamate, which acts as an intracellular store for both energy formation and provision of precursors for biosynthesis. Mouse peritoneal macrophages have been shown to utilize a high amount of glutamine via the process of glutaminolysis even though they are seen as terminally differentiated cells [21, 22]. These macrophages are characterized by high rate of protein secretion and membrane recycling [23, 24]. Although glutamine constitutes >50% of the unbound amino acid pool in human skeletal muscle, rapid reduction in blood and tissue glutamine has been noted following catabolic events such as major surgery [32], trauma [33], and sepsis [34, 35].

Glutamine is safe, well absorbed, and has no documented side effects. Glutamine is known to enhance wound healing. Glutamine and its dipeptides have been used for parenteral and enteral supplementation components in critically ill patients. A recent study by Fukuzawa, et al. [36] concluded that glutamine enhances both phagocytosis and the production of Reactive Oxygen Intermediates (ROI) by neutrophils in post operative patients. In a randomized prospective study, Morlion et al. using glutamine dipeptides in total parenteral nutrition (TPN) concluded that the supplement group had shorter hospital stay, improved immune status and nitrogen balance after abdominal surgery [37].

Alanyl-glutamine and glycyl-glutamine are two dipeptides of glutamine which have been employed clinically due to their higher solubility and chemical stability over free glutamine, making them more stable sources of the constituent amino acids [37-42]. Enteral supplementation with alanyl-glutamine but not glutamine+alanine mixture promotes intestinal adaptation as evidenced by increased peptide transport after intestinal resection [43]. Alanyl-glutamine also prevents intestinal damage, as demonstrated by increased peptide transport expression and an elevated plasma glutamine concentration after CPM administration [44]. Alanyl-glutamine alone was recently used enterally in post-operative patients for the first time with reported success and safety [53].

### SUMMARY OF THE INVENTION

We have found that intraperitoneal administration of a glutamine source will reduce post-operative adhesion formation. This is surprising because glutamine is a direct fuel for macrophages. In view of the presumed role of macrophages derived from activated milky spots in peritoneal repair and adhesion formation, one may expect that glutamine could exacerbate the formation of adhesions. While we do not wish to be bound to a particular line of reasoning with respect to an underlying mechanism pertaining to this invention, we believe that intraperitoneal administration of glutamine enhances peritoneal repair without assistance of macrophages derived from activated milky spots.

Previous therapies involving glutamine administration have employed either enteral administration (e.g. in a food supplement) or parenteral administration through the intravenous route (e.g. administration of a total parenteral nutrition formulations). As a result of this invention, it will now be known to be advantageous to employ intraperitoneal administration of a glutamine source for patients undergoing or having undergone a surgical procedure that affects the peritoneum.

The applicatants' copending application, now published as WO 2007016791 and describes formulations that act as a glutamine source and are thickened so as to have a greater viscosity than formulations which are suitable for intravenous injection. This will facilitate directed placement of the formulation within the peritoneal cavity and adherence to selected areas. Such thickened formulations must be sterile and otherwise suitable for intra-peritoneal administration and thus are different from glutamine containing formulations previously used for enteral feeding. An example is a gel containing an aqueous phase in which a glutamine source is dissolved.

A glutamine source may be administered directly to the peritoneum or within the peritoneal cavity and may also be applied to implants and/or medical devices which are placed within or near the peritoneum. Examples of such implants or medical devices include woven and non-woven resorbable materials, textiles, mesh and other barriers or shields currently used or proposed for use as means to reduce surgical adhesions. A glutamine source may be placed on, impregnated in or otherwise complexed with a surgical material or an implantable object.

In a first aspect, this invention provides compositions and products for intraperitoneal administration or placement comprising at least one glutamine source. Such a composition or product is useful for treatment of a patient to reduce post-operative adhesion formation. The composition may be present in a delivery device suitable for delivery of the composition intraperitoneally during surgery, after surgery or both.

In another aspect, this invention provides use of a glutamine source for reducing post-operative adhesion formation.

In another aspect, this invention provides use of a glutamine source in the manufacture of a medicament for intraperitoneal administration to reduce post-operative adhesion formation.

In another aspect, this invention provides a method of treating a patient to reduce post-operative adhesion formation comprising intraperitoneal administration of an effective amount of a glutamine source to said patient.

Various embodiments of this invention provide sterile compositions and products for use in reducing post-operative adhesions comprising a glutamine source for intraperitoneal administration. The composition or product may comprise one or more pharmaceutically/physiologically acceptable carriers, delivery vehicles, diluents, excipients, thickeners, viscosity enhancing agents, etc.

A viscosity enhancing agent used in this invention may form a gel when hydrated. Such gel compositions may comprise a water soluble glutamine source and a suitable gel forming agent in dry form. The latter formulation may also be provided in a partially hydrated or fully hydrated form.

This invention also provides devices suitable for performing intraperitoneal administration of a glutamine containing formulation. The device may be adapted for intra-peritoneal administration of the formulation in a variety of known ways, including by injection or other extrusion process, dropping or spraying and may take the form of a syringe, bellows container, squeezable container, pressure operated spray apparatus and the like. Also included are pumps for continuous infusion during or post-surgery, including implantable pumps.

In another aspect of the present invention, there is provided a composition for intraperitoneal administration comprising a pharmaceutically acceptable sustained release carrier and at least one glutamine source.

In another aspect of the present invention, there is provided an erosion controlled sustained release composition comprising a pharmaceutically acceptable sustained release carrier and at least one glutamine source.

In another aspect of the present invention, there is provided a composition described herein, wherein the pharmaceutically acceptable sustained release carrier is conjugated to the at least one glutamine source.

In another aspect of the present invention, there is provided a composition described herein, wherein the pharmaceutically acceptable sustained release carrier is encapsulating the at least one glutamine source.

In another aspect of the present invention, there is provided a composition described herein, wherein the pharmaceutically acceptable sustained release carrier is a synthetic polymer delivery vehicle.

In another aspect of the present invention, there is provided a composition described herein, wherein the pharmaceutically acceptable sustained release carrier is a lipid delivery vehicle.

In another aspect of the present invention, there is provided a composition described herein, wherein the lipid delivery vehicle is a 1,2-dioleoyl-1-3-trimethylammonium-propane salt.

In another aspect of the present invention, there is provided a composition described herein, wherein the pharmaceutically acceptable sustained release carrier is gelatin.

In another aspect of the present invention, there is provided a composition described herein, wherein the gelatin is in the form of a gelatin capsule.

In another aspect of the present invention, there is provided a composition described herein, wherein the pharmaceutically acceptable sustained release carrier is a mesh.

In another aspect of the present invention, there is provided a composition described herein, wherein the pharmaceutically acceptable sustained release carrier is carboxymethyl cellulose.

In another aspect of the present invention, there is provided a composition described herein, wherein the carboxymethyl cellulose is in the form of a mesh.

In another aspect of the present invention, there is provided a composition described herein, wherein the glutamine source is provided at a concentration of between about 2 g/kg to about 0.25 g/kg.

In another aspect of the present invention, there is provided a composition described herein, wherein the glutamine source is provided at a concentration of between about 1 g/kg to about 0.3 g/kg.

In another aspect of the present invention, there is provided a composition described herein, wherein the glutamine source is provided at a concentration of about 1 g/kg.

In another aspect of the present invention, there is provided a composition described herein, wherein the glutamine source is provided at a concentration of about 0.5 g/kg.

In another aspect of the present invention, there is provided a composition described herein, wherein the glutamine source is provided at a concentration of about 0.3 g/kg.

In another aspect of the present invention, there is provided a composition described herein, wherein the at least one glutamine source is a soluble peptide containing L-glutamine.

In another aspect of the present invention, there is provided a composition described herein, wherein the peptide is a dipeptide.

In another aspect of the present invention, there is provided a composition described herein, wherein the dipeptide is alanyl-glutamine.

In another aspect of the present invention, there is provided a composition described herein, wherein the at least one glutamine source is L-glutamine.

In another aspect of the present invention, there is provided a composition described herein, wherein the at least one glutamine source is in a solid form.

In another aspect of the present invention, there is provided a composition described herein for treatment of a patient to reduce post-operative adhesion formation.

In another aspect of the present invention, there is provided use of a composition described herein for reducing post-operative adhesion formation.

In another aspect of the present invention, there is provided use of a composition described herein in the manufacture of a medicament for intraperitoneal administration to reduce post-operative adhesion formation.

In another aspect of the present invention, there is provided a method of treating a patient to reduce post-operative adhesion formation comprising intraperitoneal administration of an effective amount of a composition described herein.

In another aspect of the present invention, there is provided a method described herein, wherein the composition is delivered to the peritoneal cavity during surgery.

### DETAILED DESCRIPTION OF THE INVENTION

In this specification, the term “glutamine source” includes L-glutamine and its physiologically acceptable salts, as well as peptides comprising L-glutamine. While L-glutamine may be employed in this invention, this amino acid has a relatively low water solubility (36 g/L at 20° C.) and tends to break down during sterilization and prolonged storage. Oligopeptides comprising L-glutamine which are capable of being metabolized to provide L-glutamine may also be employed in this invention. Preferably, such peptides will exhibit increased solubility in water over that of L-glutamine. Often, such peptides will also exhibit increased resistance to breakdown during sterilization and storage. Two such peptides that may be used in this invention are dipeptides comprising L-glutamine and either L-alanine or glycine. The dipeptide alanyl-glutamine (glutamine residue at the C-terminal position) has high solubility in water (568 g/L). Glycyl-glutamine (glutamine at the C-terminal position) also shows enhanced solubility in water as compared to glutamine (154 g/L). Each of the latter dipeptides is sufficiently stable during heat sterilization and prolonged storage that they have been previously employed in total parenteral nutrition formulations for intravenous injection.

Alanyl-glutamine and L-glutamine are effective in reducing and/or preventing post-operative adhesions (including secondary adhesions) when administered intra-peritoneally. This effect is not prevented by the presence of intraperitoneal bleed, suture type, or degree of inflammation. Presently, it appears the effect of alanyl-glutamine is more pronounced than that of L-glutamine in adhesion prevention.

Pharmaceutically acceptable preparations of L-glutamine and L-glutamine containing peptides (including alanyl-glutamine) are commercially available. In addition, L-glutamine containing peptides for use in this invention may also be synthesized according to known methodology and purified and sterilized for pharmaceutical use.

Compositions of this invention may be in dry, partially hydrated, or fully hydrated form and may include a glutamine source plus a pharmaceutically acceptable carrier or diluent component such as sterile distilled water, sterile isotonic solutions, sterile physiological saline solutions or dry buffer and/or salt mixes or concentrations which when diluted form such a solution. The quantity of the glutamine source in the composition may be selected in order to provide for a fully solublized amino acid or peptide during administration. Further, the quantity of L-glutamine available from the composition when formulated for administration and/or total amount of formulation administered will be selected by the skilled medical provider in order to provide a suitable dose of L-glutamine to the patient.

Based on previous studies involving intravenous administration of L-glutamine and its dipeptides, an intraperitoneal dose may provide from about 0.01 g to about 1.0 g of L-glutamine per patient kilogram, per day. However, doses may be selected to fall outside these upper and lower amounts. Typical doses employing alanyl-glutamine may be the range of about 0.1 g to about 0.5 g of the dipeptide/kg/day.

When administering a glutamine source during a surgical procedure, the typical dose for an average adult human patient may provide from about 0.3 g to about 2.0 g or about 0.3 g to about 1.5 g of L-glutamine or alanyl-glutamine within the peritoneal cavity.

Administration according to this invention may involve delivery of a glutamine source to the peritoneal cavity during surgery, at the end of surgery before closure, or soon after closure. This invention also contemplates subsequent, post-operative intraperitoneal and/or intravenous administration of a glutamine source by injection.

Formulations for use in this invention may be a solid, liquid, paste, or gel comprising a glutamine source. Compositions of this invention may be such formulations or may be compositions intended to produce such a formulation when hydrated. In a simple form, a formulation for use in this invention may consist of a glutamine source dissolved in a sterile aqueous liquid vehicle, suitable for instilling within the peritoneal cavity during surgery or for intraperitoneal injection performed thereafter. The solution may be instilled during surgery simply by injection, depositing, or spraying it into the peritoneal cavity from a suitable sterile container. The formulation may be instilled through a port created for laparoscopic surgery.

Formulations for use in this invention may be thickened in order that the formulation will exhibit increased viscosity over a typical liquid formulation suitable for intravenous injection. Such thickened formulations may be in the form of a paste or gel which may be applied directly to selected tissues or regions of the peritoneum or within the peritoneal cavity during a surgical procedure. Suitable pharmaceutically acceptable thickening agents are known and may be employed. Preferably, such an agent will form a hydrogel when hydrated or will form a hydrogel when subjected to a suitable cross-linking agent and is hydrated. Such gel forming components are selected for their biocompatibility and may be resorbable. Examples of suitable thickeners and gel forming agents which have been employed in pharmaceutical formulations include polymers having a hydrophilic component, such as collagen; polyoxyalkylene polymers such as polyethylene oxides, polyvinyl alcohols, polyvinyl pyrrolidones, and polyhydroxyethyl methacrylates; hyaluronates; and various proteins such as albumin, etc. Hemostatic gels, including those which contain fibrinogen or fibrin may also be used.

A glutamine source may also be applied to, impregnated in or delivered from implanted surgical materials or devices. For example, a gel formulation of this invention may be adhered to the exterior of an implant. Implants composed of a material such as woven resorbable cellulose (such as the kind as is sold under the trademark INTERCEED) may be impregnated with a liquid or gel formulation of this invention. Other substances for which the implanted material may be made are polyamides, polyethylenes, polypropylenes, PTFE, carbon fibers, etc.

Formulations for use in this invention may also be adapted to facilitate uptake of the glutamine source by macrophages and/or fibroblasts. In addition, or in the alternative, the formulations may provide for prolonged or delayed release of the glutamine source.

As used herein, unless otherwise specified, the term “sustained release” is used as it is generally understood in the art. For purposes of the present invention, sustained release, sustained delivery, prolonged release, and prolonged delivery, delayed release and delayed delivery are used interchangeably. A “sustained release carrier” is also used as it is generally understood in the art and is defined as a compound or composition that, when included in a composition with one or more drugs, provides a sustained release, prolonged release and/or delayed release pattern of the one or more drugs, such that the drugs are released over a period of time.

Controlled Drug Delivery: Fundamentals and Applications, Joseph Robinson et al., ((1987) ed: Robinson, pub: Informa Health Care) teaches on page 5 that “The general consensus is that controlled release denotes a system which can provide some control, whether this be of a temporal or spatial nature, or both, of drug release in the body. In other words, this system attempts to control drug concentrations in the target tissue or cells. Thus, prolonged release or sustained release systems, which only prolong therapeutic blood or tissue levels of the drug for an extended period of time, cannot be considered as controlled release systems by this definition. They are distinguished from rate-controlled drug delivery systems, which are able to specify the release rate and duration in vivo precisely, on the basis of simple in vitro tests [. . . ]. Drug targeting, on the other hand, can be considered as a form of controlled release in that it exhibits spatial control of drug release within the body. Since rate-controlled release and drug targeting represent totally separate delivery approaches, they will be discussed separately”.

Robinson et al., (supra) describes various approaches to drug release mechanisms. Robinson et al., distinguish erosion controlled approaches from diffusion controlled approaches. Erosion controlled is an approach whereby the active agent is conjugated to the sustained release agent and/or the approach whereby the active agent is surrounded by the sustained release agent and erosion of the sustained release agent provides for drug release.

Various means are available to delay or prolong delivery of a glutamine source following intraperitoneal administration. One approach is to provide the glutamine source in a solid form. At its simplest, this can involve direct administration of a solid or crystalline form of the glutamine source within the peritoneum during or at the end of surgery. Alternatively, a semi-solid or solid formulation of the glutamine source may be encapsulated and such capsule(s) placed within the peritoneum during surgery. Commonly used encapsulation materials which dissolve or otherwise degrade (e.g., through enzymatic action) include gelatin, chitosan, dextran, polymers, etc. For example, one or more gelatin capsules containing a semi-solid or solid glutamine source (e.g., powdered or crystalline L-glutamine or a glutamine peptide) may be placed within the peritoneum at the time of surgery. Alternatively, dissolving pellets may be prepared using known means (e.g., time-release pellets from Innovative Research of America, Sarasota, Fla.) to which a glutamine source is added could be employed. The glutamine source erodes, leaches or is otherwise released over time. An example of a suitable sustained release delivery vehicle is a gelatin capsules. Gelatin capsules may be prepared by inserting alanyl-glutamine and/or L-glutamine powder into a gelatin capsule and closing the gelatin capsule for use in an animal peritoneum or other closure method suitable for particular uses and/or locations.

Another known means for prolonged delivery within the peritoneum would be to adapt known techniques for continuous intraperitoneal delivery of other therapeutics such as insulin. Thus, both external and internally implantable infusion pumps, including implantable osmotic pumps may be employed for prolonged or continuous delivery of a glutamine source within the peritoneum.

Other known techniques for small molecule drug delivery may be adapted to provide for sustained or delayed release of a glutamine source within the peritoneum. A variety of delivery vehicles are known in the art that are suitable for delivery of small molecule drugs, including peptides. Generally, these involve complexing the therapeutic agent with a natural or synthetic polymer delivery vehicle; encapsulation or complexing of the therapeutic agent with a lipid delivery vehicle; or incorporation of the therapeutic agent within a three-dimensional object intended to be implanted or used as a medical device.

Examples of materials used in polymeric delivery vehicles include “natural” polymers such as collagen, fibrin, alginate, chitosan, dextran, etc., and synthetics such as silicone, polylactic acid (PLA) polymers, polyglycolide acid (PGA) polymers, polylactide-co-glycolide (PLGA) polymers, polyanhydrides, polyesters such as polycaprolactone polymers, and polyethylene polymers such as polyethylene vinyl-co-acetate (EVAc), polydioxanones, polyphosphoesters, etc. Furthermore, polymeric delivery vehicles can be adapted for covalent attachment of a peptide therapeutic agent, for example, through the use of various peptide coupling mechanisms such as carbodiimide coupling. A particularly useful coupling agent for water soluble peptide coupling applications is 1-ethyl-3(3-dimethylaminopropyl-carbodiimide) (EDC).

In addition to the gel formulations such as described above, various other structures involving the use of polymeric delivery systems are known and may be employed herein. Examples are nanospheres, microspheres, etc., prepared by complexing the glutamine source with the polymer. A variety of methods are known for preparing such formulations, including emulsion based methodology (e.g., single and double emulsions), solvent-casting approaches and foaming processes [56]. Typical procedures for collection, washing, purification, and drying may be employed, including centrifugation, filtration, lyophilization, etc. Aggregates of such compositions may also be employed.

Examples of the use of lipid based delivery vehicles include micelles and vesicles (including liposomes) in which a lipid based membrane is formed and is employed to encapsulate the glutamine source. As well, the active agent may be coupled to a lipid delivery vehicle. A variety of techniques for forming lipid delivery vehicles for delivery of peptides are well known. Furthermore, this technology has been developed to facilitate fusion of the lipid based delivery vehicle with target cells as well as preferential take-up of such a vehicle by target cells. An example would be the use of a 1,2-dioleoyl-3-trimethylammonium-propane salt (DOTAP) (on its own or in combination with other lipids such as cholesterol), to complex a glutamine source. An example would be the use of the commercial product MegaFectin™, which will be taken up by macrophages. A dipeptide such as alanyl-glutamine will be complexed with DOTAP after a short admixture period. Other examples of DOTAP preparations are known in the art [57].

As discussed above, three-dimensional products used or placed during surgery may be employed as vehicles for delivery of a glutamine source to reduce adhesion formation, particularly that which might otherwise secure the implant. Implants can provide a large depot of the glutamine source to be released over time. Structures can be formed (for example) by direct casting of a polymer and the glutamine source into a desired shape; by processing microspheres loaded with the glutamine source into a three-dimensional structure [56]; and by other known techniques. The geometry of the structure (e.g., shape, size, porosity) can be tailored to the application. For example, gas plasma may be used to provide appropriate surface features or textures to an implant. In some circumstances, it is possible to conjugate the glutamine source to the structure or scaffold of the implant providing for subsequent release of the glutamine source through endogenous cleavage mechanisms.

An example of a suitable three-dimentional structure is a mesh of carboxymethyl cellulose (CMC) mixed and/or impregnated with alanyl-glutamine and/or L-glutaminer. Such meshes may be prepared by dispersing CMC in phosphate buffer (PBS) and allowing hydration to occur. Alanyl-glutamine and/or L-glutamine may be added by mixing. The resulting mixture may be poured into a sterile Petri dish. Water is then evaporated to create a mesh. The mesh is then cross-linked and washed with 95% ethanol. The washing step may be repeated as desired. The mesh may then be dried in a biosafety hood. Appropriate shapes for the particular use of the mesh may be cut so that each shape contains a desired concentration of alanyl-glutamine and/or L-glutamine.

A glutamine source may be impregnated within a non-resorbable device, matrix or scaffold and sustained release is primarily achieved as a result of diffusion of the glutamine source from the scaffolding. An example of a non-degradable synthetic polymer that is employed in making such objects is EVAc. Resorbable structures may also be employed in which case erosion of the device itself will contribute to sustained or delayed release of the glutamine source. Examples of polymers used in the preparation of resorbable bio-materials are PGA, PLA and PLGA.

The release profile of the glutamine source from a three-dimensional structure or scaffold pursuant to this invention can be tailored through a variety of known means, including control of porosity, modification of polymer content or co-polymer ratios, etc. For example, degradation times of PLGA devices can be adapted to range from a short period to durations of months.

### EXAMPLE 1

70 plus male Wistar rats (over 350 g each) were assessed histologically on post-operative days 1, 3, 5, 7, 10, 30, and at about 6 weeks. The rats were randomly distributed based on the mode of treatment, type of sutures and presence or absence of hemorrhage during surgery. Initially, there were three surgery groups (alanyl-glutamine treatment, saline, no treatment) plus a control (no surgery). A fifth group using L-glutamine instead of alanyl-glutamine was included after preliminary results were obtained.

The rats were anaesthetized with halothane/Ketamine. Open surgery involving a midline sub-umbilical incision and a modified cecal puncture with pulstring to prevent soilage after abdominal closure was done. The procedure involved some fecal extrusion, mimicking clinical scenarios of iatrogenic/traumatic perforations of the bowel. Alanyl-glutamine [Degussa; Coubevoie, France] (0.3 g/kg-1.5 g/kg); saline (5 ml); or L-glutamine [Wiler; PCCA] (1.5 g/kg) was instilled into the peritoneal cavity from a syringe. The cecum, abdomen, and the skin were closed in layers using the same suture. Various absorbable (3/0 Vicryl™, 3/0 Monocryl™, 3/0 PDS™, 2/0 Maxon™) and non-absorbable sutures (3/0 Prolene™, 3/0 Ethibond™, 4/0 & 5/0 Surgilene™, 3/0 Novafil™) were used. The wound was infiltrated with local anesthetic. The animals were given sufficient chow to eat (20 g/day≈2 mmole/kg/day). The gastrointestinal tract and omentum were harvested from the stomach to the sigmoid colon and fixed in formaldehyde solution for evaluation.

The severity of adhesions was assessed histologically using standard Haematoxylin & Eosin and Masson trichrome stained slides. The latter helped delineate the degree of fibrosis and collagen deposition.

Semi-quantitative analysis was done by scoring the average number of milky spots per high-power-field (HPF). Quantitative statistical analysis was done using the T-test between the experimental groups. Milky spots are a marker for inflammation and adhesion formation. We found that more milky spots were associated with more adhesion formation.

On the 10^{th} post-operative day, the histological appearance of the peritoneum in alanyl-glutamine treated rats had very minimal adhesion when compared with the untreated and the saline treated surgical groups and was almost comparable to the virgin (control) rat peritoneum. This result continued in rats assessed at 6 weeks. There was 1 milky spot per 5 high power field (5HPF) in the peritoneum of the controls in comparison with 6-7 milky spots per 5HPF in the untreated surgical group. The milky spots in the alanyl-glutamine treatment group were the same as the controls while the saline treated group had 2-4 milky spots per 5HPF.

Results also revealed markedly reduced amount of acute inflammatory response in the days 3-7 (alanyl-glutamine) treatment group in comparison with the saline and non-treatment surgical groups. This was evident by the reduced amount of macrophages and macrophage chemotactic proteins. L-glutamine treatment also resulted in reduction, but to a lesser extent than in the alanyl-glutamine treated group. There was also reduced fibrosis and collagen deposition in the day-10 animals in the treatment versus saline and non-treatment surgical groups. However, saline treatment showed better results than the non-treatment surgical group.

The dosage of alanyl-glutamine was effective at 0.3 g/kg and at 1.5 g/kg. There was more adhesion from the braided sutures (3/0 Vicryl™, 2/0 Maxon™, 3/0 Dexon™ & 3/0 Ethibond™) than the non-braided or monofilaments (3/0 Monocryl™, 3/0 PDS™, 3/0 Prolene™, 4/0 & 5/0 Surgilene™, 3/0 Novafil™) in the non-treated and saline-treated groups. There also appeared to be differential tendencies towards adhesion formation with the monofilament sutures (absorbable versus non-absorbable sutures). However, alanyl-glutamine treatment prevented adhesions in all the suture groups.

Some previous human studies used parenteral doses of alanyl-glutamine ranging from 0.19-0.75 g/kg/day [42, 47]. Rats have been given 2.972 g/kg/day alanyl-glutamine and 2.0 g/kg/day L-glutamine (mixed with 1.22 g/kg/day alanine) enteral bolus supplement [43]. In the present example, we administered 0.3-1.5 g/kg alanyl-glutamine and L-glutamine intra-peritoneally. We did not observe complications due to the intraperitoneal use of alanyl-glutamine or L-glutamine as the rats recovered from surgery and continued with their usual activities.

We evaluated macrophage activity following the abdominal surgery using a macrophage chemotactic protein (MCP1) antibody stain. MCP1 is a marker for macrophage activity [46]. Our observation of reduced MCP1 in the glutamine source treatment groups as compared with the saline and non-treatment surgical groups indicated that the glutamine source treatment had an inhibitory effect on microphage migration following abdominal surgery. The fact that MCP1 was reduced by the treatment but was not completely absent is significant since complete absence is not desirable as it is associated with peritonitis and septic complications post-laparotomy [48].

Peritoneal suturing increases ischemia, devascularization, and necrosis thereby predisposing to adhesion formation [49]. Monofilaments have been shown to produce less adhesion than braided sutures because their micropores have a tendency to harbour bacteria [50, 51]. We observed a similar pattern in the untreated rats. However, the treatment prevented adhesions in all suture groups.

Hemorrhage is also associated with increased incidence of adhesion even with the use existing prevention therapies. Intraperitoneal bleeding causes intense inflammatory reactions and extensive adhesions and its relationship with adhesions is well documented in both animal and human studies [52]. We observed that hemorrhage did not prevent the effect of the treatment.

### EXAMPLE 2

Previous studies show a high incidence of adhesion formation when surgical mesh is employed as a reinforcing material [6a, 6b]. We repeated trials as described in Example 1, inserting Marlex™ mesh during the procedure on the surgical groups. Comparable results to that described in Example 1 were obtained between days 1-42.

Severity of adhesions was assessed at day 90 using the adhesion score of Zuhlke, et al. [54]. This scoring procedure has been described in the literature for assessment of other potential treatments for adhesions [55]. At day 90, the adhesion score in animals treated with alanyl-glutamine was 0-1 (no adhesion or flimsy adhesion), comparable to the virgin abdomens of the control group. This differs from previous measurements of adhesions using polypropylene mesh which reported up to about 90% adhesion formation [6a].

### EXAMPLE 3

Adhesions tend to recur once in place. We examined the effect of alanyl-glutamine treatment on such secondary adhesions. The modified cecal perforation with pulstring closure procedure described in Example 1 was performed on 9 Sprague-Dawley rats. These were divided into 5 groups, each group receiving a different form of mesh (INTERCEED™; PROCEED™; BARD™ composite; prolene; and a mesh which was allowed to be “infected” by repeated re-opening of the wound). The surgical procedure with mesh implacement was performed without application of a glutamine source. Six months after initial surgery, a laparotomy was performed on each rat then adhesiolysis followed by installation of alanyl-glutamine. Three weeks later the rats were assessed for recurrence of adhesions and scored according to Zuhlke, et al. [54] and no secondary adhesions were seen.

### EXAMPLE 4

15 Wistar rats greater than 300 g were subjected to cecal perforation via pulstring suturing as described above in Example 1. Each rat was also provided with a prolene mesh designed to increase the chances of adhesion formation. The rats were separated into 5 groups of three rats each:

a) rats to be administered with 2 mL of a DOTAP formulation containing a concentration of 1 g/kg of alanyl-glutamine;

b) rats to be administered with 2 mL of a DOTAP formulation containing a concentration of 0.5 g/kg of alanyl-glutamine;

c) rats to be administered with 2 mL of a DOTAP formulation containing a concentration of 0.3 g/kg of alanyl-glutamine;

d) rats to be administered with gelatin capsules containing a concentration of 1 g/kg of L-glutamine; and

e) rats to be administered with a saline rinse (control group).

The DOTAP was kindly provided by Vaccine and Infectious Disease Organization (VIDO), and is available commercially from Sigma. We added an aqueous solution containing alanyl-glutamine to the DOTAP solution to form a primary emulsion in a ratio (alanyl-glutamine:DOTAP) of 3:1 w/w, 2:1 w/w and 1:1 w/w to provide for the concentrations of 1 g/kg, 0.5 g/kg and 0.3 g/kg, respectively The DOTAP and the alanyl-glutamine were mixed together by shaking for about 1 to 2 minutes, 10 minutes prior to use. The resulting mixture is a DOTAP cationic liposome which sustains the release of alanyl-glutamine over a 4 day period.

Gelatin capsules were prepared by inserting L-glutamine powder into gelatin capsules in a pharmacy and closing the capsule for use in an animal peritoneum.

The rats were administered with the appropriate DOTAP formulation (topically to the peritoneum using a syringe), gelatin capsules (topically by hand to the peritoneum) or saline rinse (topically to the peritoneum using a syringe) according to the groupings described above. Rats from each of groups a) to e) were subjected to a laparotomy and some rats from each of groups a) to e) were euthanized at 4 and other rats from each of groups a) to e) were euthanized at 8 weeks. The rats were reopened after euthanasia and the contents of their abdomen and the anterior abdominal wall were examined.

Adhesion involves organs attaching to themselves and the anterior abdominal wall. The rats in groups a) and b) showed no adhesions and were given a Zuhlke rating score of 0. The rats in group c) showed flimsy adhesions or fine adhesions only and were given a Zuhlke rating score of 1. The rats in group d) showed fine adhesions and were given a Zuhlke rating score of 1. The rats in group e) showed significant adhesions and were given a Zuhlke rating score of 4 or 5.

### EXAMPLE 5

A mesh of carboxymethyl cellulose (CMC) and alanyl-glutamine was prepared by dispersing 0.17 g of CMC in 20 mL phosphate buffer (PBS) at pH 6.4 and allowing hydration for 1 to 2 hours. 5 mL of alanyl-glutamine was added by mixing. The mixture was poured into a 10 cm diameter sterile Petri dish. Water was evaporated at 40° C. over nitrogen to create a mesh. The mesh was cross-linked with 10 mL of 15 mM EDC for 24 hours at room temperature. The mesh was washed with 95% ethanol three times, then dried in a biosafety hood. Circles of 2.5 cm diameter were cut so that each circle contained 0.3 mL of alanyl-glutamine.

A rat was subjected to cecal perforation via pulstring suturing as described above in Example 1. The rat was then administered with a CMC mesh circle of 2.5 cm as described above. The rat was then closed up. 7 days after the cecal perforation, the rat was euthanized, reopened and the contents of the abdomen and the anterior abdominal wall were examined. A single, fine adhesion, classified as a Zuhlke score 1, of the anterior abdominal wall to the intestine was observed. The rest of the belly was free of adhesion.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

### REFERENCES

1. Thompson, J. N. and S. A. Whawell, Pathogenesis and prevention of adhesion formation. Br J Surg, 1995. 82(1): p. 3-5.

2. Thompson, J. N., Preventing adhesions. Lancet, 1995. 346(8987): p. 1382.

3. Ellis, H., The clinical significance of adhesions: focus on intestinal obstruction. Eur J Surg Suppl, 1997(577): p. 5-9.

4. Menzies, D. and H. Ellis, Intestinal obstruction from adhesions—how big is the problem? Ann R Coll Surg Engl, 1990. 72(1): p. 60-3.

5. Parker, M. C., et al., Postoperative adhesions: ten-year follow-up of 12,584 patients undergoing lower abdominal surgery. Dis Colon Rectum, 2001. 44(6): p. 822-29; discussion 829-30.

6. Ellis, H., The causes and prevention of intestinal adhesions. Br J Surg, 1982. 69(5): p. 241-3.

6a. Naim, J. O., et al., J. Laparoendosc Surg, 1993. 3: p. 187-90.

6b. Baykal, A., et al., World J Surg, 1997. 21: p. 579-82.

7. Menzies, D., Peritoneal adhesions. Incidence, cause, and prevention. Surg Annu, 1992. 24 Pt 1: p. 27-45.

8. Bridges, J. B., F. R. Johnson, and H. W. Whitting, Peritoneal adhesion formation. Acta Anat (Basel), 1965. 61(2): p. 203-12.

9. Drollette, C. M. and S. Z. Badawy, Pathophysiology of pelvic adhesions. Modern trends in preventing infertility. J Reprod Med, 1992. 37(2): p. 107-21; discussion 121-2.

10. Vural, B., et al., The role of neutrophils in the formation of peritoneal adhesions. Hum Reprod, 1999. 14(1): p. 49-54.

11. Ackermann, P. C., P. D. De Wet, and G. P. Loots, Microcirculation of the rat omentum studied by means of corrosion casts. Acta Anat (Basel), 1991. 140(2): p. 146-9.

12. Shimotsuma, M., et al., Ontogeny of milky spots in the fetal lamb omentum. Arch Histol Cytol, 1994. 57(3): p. 291-9.

13. Takemori, N., [Morphological studies of the omental milk spots in the mouse: light and electron microscopy (author's transl)]. Hokkaido Igaku Zasshi, 1979. 54(3): p. 265-83.

14. Shimotsuma, M., et al., Milky spots in the human greater omentum. Macroscopic and histological identification. Acta Anat (Basel), 1989. 136(3): p. 211-6.

15. Shimotsuma, M., et al., Cellular subsets of the milky spots in the human greater omentum. Cell Tissue Res, 1991. 264(3): p. 599-601.

16. Williams, R. and H. White, The greater omentum: its applicability to cancer surgery and cancer therapy. Curr Probl Surg, 1986. 23(11): p. 789-865.

17. Dux, K., Proliferative activity of macrophages in the greater omentum of the mouse in relation to the early postnatal development of the vascular structures. J Leukoc Biol, 1986. 40(4): p. 445-58.

18. Rodgers, K. E. and G. S. diZerega, Function of peritoneal exudate cells after abdominal surgery. J Invest Surg, 1993. 6(1): p. 9-23.

19. Cranshaw, M. L. and L. V. Leak, Milky spots of the omentum: a source of peritoneal cells in the normal and stimulated animal. Arch Histol Cytol, 1990. 53 Suppl: p. 165-77.

20. Newsholme, E. A., The possible role of glutamine in some cells of the immune system and the possible consequence for the whole animal. Experientia, 1996. 52(5): p. 455-9.

21. Newsholme, E. A., B. Crabtree, and M. S. Ardawi, The role of high rates of glycolysis and glutamine utilization in rapidly dividing cells. Biosci Rep, 1985. 5(5): p. 393-400.

22. Newsholme, E. A., B. Crabtree, and M. S. Ardawi, Glutamine metabolism in lymphocytes: its biochemical, physiological and clinical importance. Q J Exp Physiol, 1985. 70(4): p. 473-89.

23. Werb, Z. and Z. A. Cohn, Plasma membrane synthesis in the macrophage following phagocytosis of polystyrene latex particles. J Biol Chem, 1972. 247(8): p. 2439-46.

24. Ardawi, M. S. and E. A. Newsholme, Glutamine metabolism in lymphocytes of the rat. Biochem J, 1983. 212(3): p. 835-42.

25. Dijkstra, F. R., et al., Recent clinical developments in pathophysiology, epidemiology, diagnosis and treatment of intra-abdominal adhesions. Scand J Gastroenterol Suppl, 2000(232): p. 52-9.

26. Vipond, M. N., et al., Peritoneal fibrinolytic activity and intra-abdominal adhesions. Lancet, 1990. 335(8698): p. 1120-2.

27. Whawell, S. A. and J. N. Thompson, Cytokine-induced release of plasminogen activator inhibitor-1 by human mesothelial cells. Eur J Surg, 1995. 161(5): p. 315-8.

28. Holmdahl, L. and M. L. Ivarsson, The role of cytokines, coagulation, and fibrinolysis in peritoneal tissue repair. Eur J Surg, 1999. 165(11): p. 1012-9.

29. Ivarsson, M. L., et al., Tissue markers as predictors of postoperative adhesions. Br J Surg, 1998. 85(11): p. 1549-54.

30. Smith, R. J., Glutamine metabolism and its physiologic importance. JPEN J Parenter Enteral Nutr, 1990. 14(4 Suppl): p. 40S-44S.

31. Lacey, J. M. and D. W. Wilmore, Is glutamine a conditionally essential amino acid? Nutr Rev, 1990. 48(8): p. 297-309.

32. Vinnars, E., J. Bergstom, and P. Furst, Influence of the postoperative state on the intracellular free amino acids in human muscle tissue. Ann Surg, 1975. 182(6): p. 665-71.

33. Askanazi, J., et al., Muscle and plasma amino acids after injury: hypocaloric glucose vs. amino acid infusion. Ann Surg, 1980. 191(4): p. 465-72.

34. Roth, E., [Changes in protein metabolism in cachexia and catabolism]. Z Exp Chir Transplant Kunstliche Organe, 1985. 18(3): p. 150-6.

35. Roth, E., et al., Liver amino acids in sepsis. Surgery, 1985. 97(4): p. 436-42.

36. Fukuzawa, K., et al., N-acetylcysteine ameliorates reperfusion injury after warm hepatic ischemia. Transplantation, 1995. 59(1): p. 6-9.

37. Morlion, B. J., et al., Total parenteral nutrition with glutamine dipeptide after major abdominal surgery: a randomized, double-blind, controlled study. Ann Surg, 1998. 227(2): p. 302-8.

38. Furst, P., S. Albers, and P. Stehle, Availability of glutamine supplied intravenously as alanylglutamine. Metabolism, 1989. 38(8 Suppl 1): p. 67-72.

39. Kamer, J. and E. Roth, Influence of alanylglutamine infusion on gastrointestinal glutamine and alanine metabolism in anesthetized dogs. Metabolism, 1989. 38(8 Suppl 1): p. 73-7.

40. Babst, R., et al., Glutamine peptide-supplemented long-term total parenteral nutrition: effects on intracellular and extracellular amino acid patterns, nitrogen economy, and tissue morphology in growing rats. JPEN J Parenter Enteral Nutr, 1993. 17(6): p. 566-74.

41. Nordfjeld, K., M. Rasmussen, and V. G. Jensen, Storage of mixtures for total parenteral nutrition—long-term stability of a total parenteral nutrition mixture. J Clin Hosp Pharm, 1983. 8(3): p. 265-74.

42. Cardona Pera, D., [Administration of glutamine and its dipeptides in parenteral nutrition. Which patients are candidates?]. Nutr Hosp, 1998. 13(1): p. 8-20.

43. Satoh, J., et al., Enteral alanyl-glutamine supplement promotes intestinal adaptation in rats. Int J Mol Med, 2003. 12(4): p. 615-20.

44. Satoh, J., et al., Nutritional benefits of enteral alanyl-glutamine supplementation on rat small intestinal damage induced by cyclophosphamide. J Gastroenterol Hepatol, 2003. 18(6): p. 719-25.

45. Ray, N. F., et al., Abdominal adhesiolysis: inpatient care and expenditures in the United States in 1994. J Am Coll Surg, 1998. 186(1): p. 1-9.

46. Valente, A. J., et al., Mechanisms in intimal monocyte-macrophage recruitment. A special role for monocyte chemotactic protein-1. Circulation, 1992. 86(6 Suppl): p. III20-5.

47. Ward, E., et al., Oral glutamine in paediatric oncology patients: a dose finding study. Eur J Clin Nutr, 2003. 57(1): p. 31-6.

48. Matsukawa, A., et al., Endogenous monocyte chemoattractant protein-1 (MCP-1) protects mice in a model of acute septic peritonitis: cross-talk between MCP-1 and leukotriene B4. J Immunol, 1999. 163(11): p. 6148-54.

49. Luijendijk, R. W., et al., Foreign material in postoperative adhesions. Ann Surg, 1996. 223(3): p. 242-8.

50. Liakakos, T., et al., Peritoneal adhesions: etiology, pathophysiology, and clinical significance. Recent advances in prevention and management. Dig Surg, 2001. 18(4): p. 260-73.

51. Bakkum, E. A., et al., Quantitative analysis of the inflammatory reaction surrounding sutures commonly used in operative procedures and the relation to postsurgical adhesion formation. Biomaterials, 1995. 16(17): p. 1283-9.

52. Gadallah, M. F., et al., Relationship between intraperitoneal bleeding, adhesions, and peritoneal dialysis catheter failure: a method of prevention. Adv Petit Dial, 2001. 17: p. 127-9.

53. Obayan, A. O. E., Oxidative stress: Natural History and Modulation in Surgery and Trauma Patients. Ph.D. in Surgery Thesis, University of Saskatchewan, Spring 2004.

54. Zuhlke, H. V., et al., Langenbecks Arch Chir Suppl II Verh Dtsch Ges Chir, 1990. p. 1009-16.

55. Bae, J. S., et al., World J Gastroenterol 11: p. 810-816.

56. Harris, L. D., et al., J. Biomed. Mater. Res. (1998). 42:396-402.

57. Heil, et al. Science (2004). 303:1526-1529.

1. A composition for intraperitoneal administration comprising a pharmaceutically acceptable sustained release carrier and at least one glutamine source.

2. The composition of claim 1, wherein the composition is configured to be an erosion controlled sustained release composition.

3. The composition of claim 1, wherein the pharmaceutically acceptable sustained release carrier is conjugated to the at least one glutamine source.

4. The composition of claim 1, wherein the pharmaceutically acceptable sustained release carrier is encapsulating the at least one glutamine source.

5. The composition of any one of claim 1, wherein the pharmaceutically acceptable sustained release carrier is a synthetic polymer delivery vehicle.

6. The composition of any one of claim 1, wherein the pharmaceutically acceptable sustained release carrier is a lipid delivery vehicle.

7. The composition of claim 6, wherein the lipid delivery vehicle is a 1,2-dioleoyl-1-3-trimethylammonium-propane salt.

8. The composition of claim 1, wherein the pharmaceutically acceptable sustained release carrier is gelatin.

9. The composition of claim 8, wherein the gelatin is in the form of a gelatin capsule.

10. The composition of any one of claim 1, wherein the pharmaceutically acceptable sustained release carrier is a mesh.

11. The composition of claim 1, wherein the pharmaceutically acceptable sustained release carrier is carboxymethyl cellulose.

12. The composition of claim 11, wherein the carboxymethyl cellulose is in the form of a mesh.

13. The composition of claim 1, wherein the glutamine source is provided at a concentration of between about 2 g/kg to about 0.25 g/kg.

14. The composition of any one of claim 1, wherein the glutamine source is provided at a concentration of between about 1 g/kg to about 0.3 g/kg.

15. The composition of any one of claim 1, wherein the glutamine source is provided at a concentration of about 1 g/kg.

16. The composition of any one of claim 1, wherein the glutamine source is provided at a concentration of about 0.5 g/kg.

17. The composition of any one of claim 1, wherein the glutamine source is provided at a concentration of about 0.3 g/kg.

18. The composition of any one of claim 1, wherein the at least one glutamine source is a soluble peptide containing L-glutamine.

19. The composition of claim 18, wherein the peptide is a dipeptide.

20. The composition of claim 19, wherein the dipeptide is alanyl-glutamine.

21. The composition of any one of claim 1, wherein the at least one glutamine source is L-glutamine.

22. The composition of any one of claim 1, wherein the at least one glutamine source is in a solid form.

23. The composition of any one of claim 1, for treatment of a patient to reduce post-operative adhesion formation.

24. (canceled)

25. (canceled)

26. A method of treating a patient to reduce post-operative adhesion formation comprising intraperitoneal administration of an effective amount of a composition according to claim 1 to said patient.

27. The method of claim 26, wherein the composition is delivered to the peritoneal cavity during surgery.
